(19) United States
(12) Patent Application Publication
(10) Pub. No.: US 2023/0069870 A1
(43) Pub. Date: Mar. 9, 2023

(54) ULTRASOUND SIGNAL PROCESSING APPARATUS, METHOD OF OPERATING ULTRASOUND SIGNAL PROCESSING APPARATUS, AND COMPUTER-READABLE RECORDING MEDIUM

(71) Applicant: OLYMPUS CORPORATION, Tokyo (JP)

(72) Inventor: Kazuhito NEMOTO, Tokyo (JP)

(73) Assignee: OLYMPUS CORPORATION, Tokyo (JP)

(21) Appl. No.: 17/978,380

(22) Filed: Nov. 1, 2022

Related U.S. Application Data
(63) Continuation of application No. PCT/JP2020/021243, filed on May 28, 2020.

(51) Int. Cl. G01S 7/52; A61B 8/08; A61B 8/00; G01S 15/89

(52) U.S. Cl. CPC G01S 7/52085; A61B 8/5207; A61B 8/54; G01S 15/8906; G01S 7/52028; A61B 8/12

(57) ABSTRACT

An ultrasound signal processing apparatus includes: a receiver configured to receive a positive phase ultrasound reception signal and a negative phase ultrasound reception signal; and a processor including hardware, the processor being configured to perform phasing addition on each of the positive phase ultrasound reception signal and the negative phase ultrasound reception signal, and adding the positive phase ultrasound reception signal subjected to the phasing addition and the negative phase ultrasound reception signal subjected to the phasing addition with shifting of a predetermined period of time.

### CROSS-REFERENCE TO RELATED APPLICATION

This application is a continuation of International Application No. PCT/JP2020/021243, filed on May 28, 2020, the entire contents of which are incorporated herein by reference.

### BACKGROUND

### 1. Technical Field

The present disclosure relates to an ultrasound signal processing apparatus, a method of operating the ultrasound signal processing apparatus, and a computer-readable recording medium.

### 2. Related Art

In the related art, in an ultrasound observation technology, in some cases, an ultrasound image is generated by using a harmonic component that is generated from ultrasound waves (fundamental waves) propagating through an inside of a living body. In addition, there is a known pulse inversion technique for irradiating the inside of the living body with a positive phase ultrasound pulse and a negative phase ultrasound pulse, removing a fundamental wave component by adding a positive phase echo signal and a negative phase echo signal that are reflected inside the living body, and extracting only the harmonic component.

Incidentally, if a positive phase and a negative phase are asymmetric, it is not possible to remove the fundamental wave component even if the echo signals are added, and thus, it is not possible to appropriately extract the harmonic component. In order to solve this problem, Japanese Patent No. 5337836 discloses a technology for appropriately extracting a harmonic component by using a gain or a filter.

### SUMMARY

In some embodiments, an ultrasound signal processing apparatus includes: a receiver configured to receive a positive phase ultrasound reception signal and a negative phase ultrasound reception signal; and a processor including hardware, the processor being configured to perform phasing addition on each of the positive phase ultrasound reception signal and the negative phase ultrasound reception signal, and adding the positive phase ultrasound reception signal subjected to the phasing addition and the negative phase ultrasound reception signal subjected to the phasing addition with shifting of a predetermined period of time.

In some embodiments, provided is a method of operating an ultrasound signal processing apparatus. The method includes: receiving, by a receiver, a positive phase ultrasound reception signal and a negative phase ultrasound reception signal; performing, by a processor including hardware, phasing addition on each of the positive phase ultrasound reception signal and the negative phase ultrasound reception signal; and adding, by the processor, the positive phase ultrasound reception signal subjected to the phasing addition and the negative phase ultrasound reception signal subjected to the phasing addition with shifting of a predetermined period of time.

In some embodiments, provided is a non-transitory computer-readable recording medium with an executable program stored thereon. The program causes an ultrasound signal processing apparatus to execute: receiving, by a receiver, a positive phase ultrasound reception signal and a negative phase ultrasound reception signal; performing, by a processor including hardware, phasing addition on each of the positive phase ultrasound reception signal and the negative phase ultrasound reception signal; and adding, by the processor, the positive phase ultrasound reception signal subjected to the phasing addition and the negative phase ultrasound reception signal subjected to the phasing addition with shifting of a predetermined period of time. The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating the overall configuration of an endoscope system including an ultrasound signal processing apparatus according to a first embodiment;

FIG. 2 is a block diagram illustrating a configuration of the endoscope system including the ultrasound signal processing apparatus according to the first embodiment;

FIG. 3 is a diagram illustrating a process performed on an echo signal;

FIG. 4 is a diagram illustrating a commonly performed process for adding a positive phase ultrasound reception signal and a negative phase ultrasound reception signal;

FIG. 5 is a diagram illustrating an addition result that is obtained by changing a shift amount of a period of time between positive phase ultrasound echoes and negative phase ultrasound echoes and that is represented by using a frequency component;

FIG. 6 is a diagram illustrating a relationship between a depth and an intensity of each of a fundamental wave component and a harmonic component and a relationship between a depth and a shift amount;

FIG. 7 is a flowchart illustrating an outline of a process performed by the ultrasound signal processing apparatus according to the first embodiment;

FIG. 8 is a diagram illustrating an example of an input of an observation position; and FIG. 9 is a diagram illustrating one example of an ultrasound image.

### DETAILED DESCRIPTION

Preferred embodiments of an ultrasound signal processing apparatus, a method of operating the ultrasound signal processing apparatus, and a program for operating the ultrasound signal processing apparatus according to the disclosure will be explained below with reference to accompanying drawings. Furthermore, the disclosure is not limited to the embodiments below. The disclosure is applicable to an ultrasound signal processing apparatus that performs phasing addition, a method of operating the ultrasound signal processing apparatus, and a program for operating the ultrasound signal processing apparatus that are typically used.

Furthermore, in the descriptions of the drawings, components that are identical or corresponding to those in embodiments are appropriately denoted by the same reference numerals. In addition, it is necessary to note that the drawings used for the descriptions below are only schematic illustrations and the relationship of the size among the components, the ratios of the components, and the like may be different from those used in practice. Moreover, the drawings may include portions in which the relationship of the size among the components and the ratios of the components may sometimes differ between the drawings.

### First Embodiment

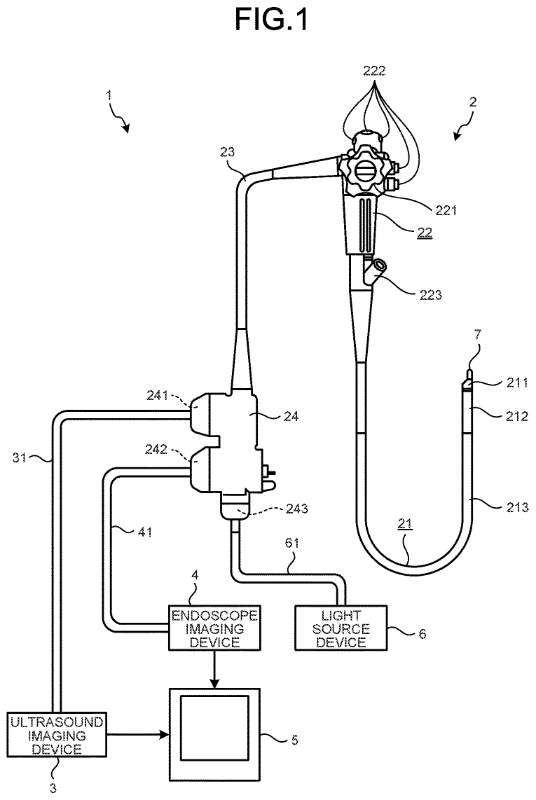

Configuration of endoscope system FIG. 1 is a diagram schematically illustrating the entirety of an endoscope system including an ultrasound signal processing apparatus according to a first embodiment. An endoscope system 1 is a system that performs ultrasound wave observation in an inside of a subject, such as a person, by using an ultrasound endoscope. The endoscope system 1 includes, as illustrated in FIG. 1, an ultrasound endoscope 2, an ultrasound imaging device 3 that is an ultrasound signal processing apparatus, an endoscope imaging device 4, a display device 5, a light source device 6, and an ultrasound transducer 7.

The ultrasound endoscope 2 includes the ultrasound transducer 7 at a distal end portion of the ultrasound endoscope 2, converts an electrical pulse signal (hereinafter, also referred to as an “ultrasound transmission signal”) transmitted from the ultrasound imaging device 3 to an ultrasound pulse (acoustic pulse), and irradiates the subject with the converted ultrasound pulse, and then, converts ultrasound echoes reflected at the subject to an electrical echo signal (hereinafter, also referred to as an “ultrasound reception signal”) that represents using a voltage change and outputs the echo signal.

The ultrasound endoscope 2 generally includes an imaging optical system and an image sensor, is inserted into the digestive tract (an esophagus, a stomach, a duodenum, or a large intestine) or the respiratory organs (a trachea or a bronchus) of the subject, and is able to capture images of the digestive tracts or the respiratory organs. Furthermore, the ultrasound endoscope 2 is able to capture images of the surrounding organs (a pancreas, a gallbladder, a bile duct, a biliary tract, a lymph node, a mediastinal organ, a blood vessel, etc.) by using ultrasound waves. In addition, the ultrasound endoscope 2 includes a light guide that introduces illumination light for irradiating subject with light at the time of optical image capturing. The distal end portion of the light guide reaches the distal end of the insertion portion of the ultrasound endoscope 2 with respect to the subject, whereas the proximal end portion of the light guide is connected to the light source device 6 that generates illumination light.

The ultrasound endoscope 2 includes, as illustrated in FIG. 1, an insertion portion 21, an operating unit 22, a universal cord 23, and a connector 24. The insertion portion 21 is a portion that is inserted into the subject.

The insertion portion 21 is provided, as illustrated in FIG. 1, on the distal end side, and includes a rigid distal end hard portion 211 that holds the ultrasound transducer 7 that transmits and receives ultrasound waves, a bendable portion 212 that is coupled to the proximal end side of the distal end hard portion 211 and that is freely bendable, and a flexible tube portion 213 that is coupled to the proximal end side of the bendable portion 212 and that has flexibility. Here, although not illustrated, in the interior portion of the insertion portion 21, the light guide that transmits illumination light supplied from the light source device 6 and a plurality of signal cables that transmit various signals are wired, and a treatment instrument insertion port for inserting a treatment instrument is formed. Furthermore, in the present specification, the insertion portion 21 on the ultrasound transducer 7 side is referred to as a distal end side, whereas the insertion portion 21 on the side that is coupled to the operating unit 22 is referred to as a proximal end side.

The operating unit 22 is a portion that is coupled to the proximal end side of the insertion portion 21 and receives various operations from a doctor or the like. The operating unit 22 includes, as illustrated in FIG. 1, a bending knob 221 that allows the bendable portion 212 to perform bending operation, and a plurality of operating members 222 that are used for various operations.

Furthermore, the operating unit 22 is provided with a treatment instrument insertion port 223 that communicates with the treatment instrument insertion port and that allows the treatment instrument to insert in the treatment instrument insertion port.

The universal cord 23 is a cable that extends from the operating unit 22, and in which a plurality of signal cables that transmit various signals, an optical fiber that transmits the illumination light supplied from the light source device 6, and the like are disposed.

The connector 24 is provided at the distal end of the universal cord 23. In addition, the connector 24 includes first to third connector portions 241 to 243 to which an ultrasound cable 31, a video cable 41, and an optical fiber cable 61 are connected, respectively.

The ultrasound imaging device 3 is electrically connected to the ultrasound endoscope 2 via the ultrasound cable 31 (see FIG. 1), outputs an ultrasound transmission signal that is a pulse signal to the ultrasound endoscope 2 via the ultrasound cable 31, and inputs an echo signal from the ultrasound endoscope 2. Then, the ultrasound imaging device 3 generates an ultrasound image by performing a predetermined process on the echo signal.

The endoscope imaging device 4 is electrically connected to the ultrasound endoscope 2 via the video cable 41 (see FIG. 1), and inputs an image signal received from the ultrasound endoscope 2 via the video cable 41. Then, the endoscope imaging device 4 generates an endoscope image by performing a predetermined process on the image signal.

The display device 5 is constituted by using a liquid crystal or organic electro luminescence (EL), a projector, a cathode ray tube (CRT), or the like, and displays an ultrasound image generated by the ultrasound imaging device 3, an endoscope image generated by the endoscope imaging device 4, or the like.

The light source device 6 is connected to the ultrasound endoscope 2 via the optical fiber cable 61 (FIG. 1), and supplies illumination light for illuminating the interior portion of the subject via the optical fiber cable 61 to the ultrasound endoscope 2.

Any scanning type including a convex type, a linear type, a radial type, or the like may be used for the ultrasound transducer 7. The ultrasound endoscope 2 may allow the ultrasound transducer 7 to mechanically perform scanning, or may provide a plurality of arrayed piezoelectric elements as the ultrasound transducer 7 and electronically perform scanning by electronically switching the piezoelectric elements involved in transmission and reception operations or by adding a delay to the transmission and reception operations performed by each of the piezoelectric elements. In addition, the ultrasound transducer 7 may be two-dimensionally arrayed piezoelectric elements.

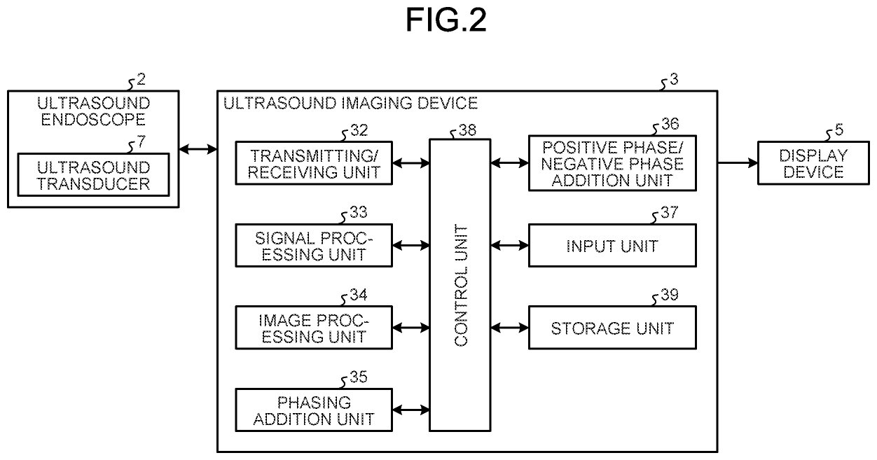

FIG. 2 is a block diagram illustrating a configuration of an endoscope system including the ultrasound signal processing apparatus according to the first embodiment.

The ultrasound imaging device 3 includes, as illustrated in FIG. 2, a transmitting/receiving unit 32, a signal processing unit 33, an image processing unit 34, a phasing addition unit 35, a positive phase/negative phase addition unit 36, an input unit 37, a control unit 38, and a storage unit 39.

The transmitting/receiving unit 32 transmits and receives, under the control of the control unit 38, positive phase signal and a negative phase signal to and from the plurality of piezoelectric elements that are included in the ultrasound transducer 7. Specifically, the transmitting/receiving unit 32 includes a transmitting unit that is electrically connected to the ultrasound endoscope 2, and that transmits, to the ultrasound transducer 7, an ultrasound transmission signal constituted of a high voltage pulse on the basis of a predetermined waveform and transmission timing. Furthermore, the transmitting/receiving unit 32 includes a receiving unit that receives an electrical echo signal from the ultrasound transducer 7, that generates digital data on a high radio frequency (RF) signal (hereinafter, referred to as “RF data”), and that outputs the generated data. It is preferable that the frequency band of the pulse signal transmitted by the transmitting/receiving unit 32 includes, in a linear response frequency band for electro-acoustic conversion to be subjected to a pulse signal that is output from the ultrasound transducer 7 with respect to an ultrasound pulse, the linear response frequency band and a harmonic band that is generated from the conversion. In addition, the transmitting/receiving unit 32 also has a digital data communication function for transmitting various control signals output from the control unit 38 to the ultrasound endoscope 2, receiving various kinds of digital data including an ID that is used for identification from the ultrasound endoscope 2, and transmitting the received digital data to the control unit 38.

The signal processing unit 33 generates digital B mode purpose reception data on the basis of synthetic output data (an ultrasound reception signal that is subjected to phasing addition, or, a synthetic signal that is subjected to addition of a positive phase and a negative phase) that is input from the positive phase/negative phase addition unit 36 (or, the phasing addition unit 35). Specifically, the signal processing unit 33 performs a known process, such as a bandpass filter process, envelope detection, or logarithmic transformation, on the synthetic output data, and generates the digital B mode purpose reception data.

In the logarithmic transformation, common logarithms obtained by dividing the synthetic output data by a reference voltage Ve is represented by a decibel value.

The signal processing unit 33 outputs the generated B mode purpose reception data corresponding to a single frame to the image processing unit 34. The signal processing unit 33 is implemented by a central processing unit (CPU), various arithmetic circuits, or the like.

The image processing unit 34 generates image data on the basis of the B mode purpose reception data input from the signal processing unit 33. The image processing unit 34 performs signal processing on the B mode purpose reception data received from the signal processing unit 33 by using a known technology, such as a scan converter process, a gain process, or a contrast process. In the scan converter process, a scanning direction of the B mode purpose reception data is converted from the scanning direction of the ultrasound waves to a display direction of the display device 5. The B mode image is a grayscale image in which values of R (red), G (green), and B (blue) that are variables in the case in which an RGB color system is used as a color space are matched. Furthermore, the image processing unit 34 performs coordinate conversion on the B mode purpose reception data received from the signal processing unit 33 such that the scanning range is rearranged so as to be spatially and correctly represented, and then, fills a vacant space between the pieces of B mode purpose reception data by performing an interpolation process between the pieces of B mode purpose reception data, and generates the B mode image data that is in accordance with the display range of the image displayed on the display device 5. The image processing unit 34 is implemented by using a CPU, various arithmetic circuits, or the like.

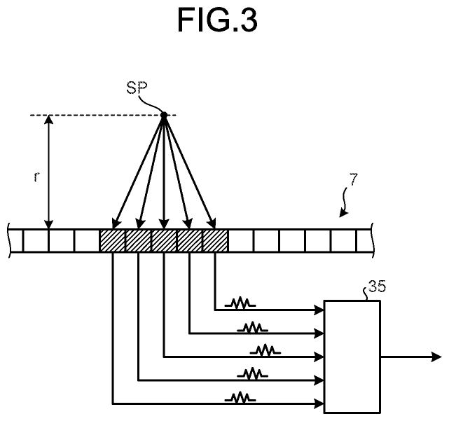

The phasing addition unit 35 performs phasing addition on the RF data the tis input from the transmitting/receiving unit 32. FIG. 3 is a diagram illustrating a process performed on an echo signal. As illustrated in FIG. 3, each of the piezoelectric elements included in the ultrasound transducer 7 receives an echo signal in a state in which each of the echo signals is temporally shifted in accordance with a distance r from a sampling point SP. The received echo signals are output as RF data from the transmitting/receiving unit 32, and are input to the phasing addition unit 35 after being temporarily stored in the storage unit 39. The phasing addition unit 35 adds an appropriate delay to the RF data in accordance with the distance r, performs addition by aligning the phases, and obtains an ultrasound reception signal that has been subjected to phasing addition. The phasing addition unit 35 is implemented by using a CPU, various arithmetic circuits, or the like.

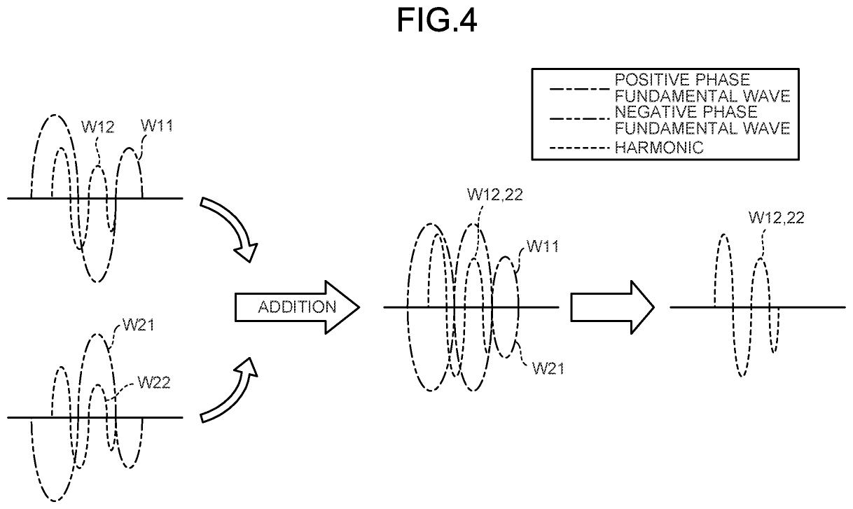

The positive phase/negative phase addition unit 36 obtains a synthetic signal (hereinafter, also referred to as a “synthetic wave”) that is obtained by adding a positive phase ultrasound reception signal that is subjected to phasing addition and a negative phase ultrasound reception signal that is subjected to phasing addition after the timing of both of the ultrasound reception signals are made to coincide. FIG. 4 is a diagram illustrating a general state in which a positive phase ultrasound reception signal and a negative phase ultrasound reception signal are added. The vertical axis illustrated in FIG. 4 indicates an amplitude, whereas the horizontal axis indicates time. As illustrated in FIG. 4, if the positive phase ultrasound reception signal and the negative phase ultrasound reception signal are added, a fundamental wave W11 that is in positive phase and that is indicated by an alternate long and short dash line and a fundamental wave W21 that is in negative phase and that is indicated by a chain double-dashed line are removed by being canceled out each other. In contrast, a harmonic W12 that is in positive phase indicated by a broken line and a harmonic W22 that is in negative phase indicated by a broken line reinforce with each other, and have twice the amplitude of the fundamental waves W11 and W21. As a result, ideally, only harmonic components remain.

However, in practice, in some cases, a shift occurs between a positive phase ultrasound reception signal and a negative phase ultrasound reception signal in a time direction. For example, due to the reason that a response from a semiconductor at the time of generation of an ultrasound transmission signal is different in accordance with the polarity of the voltage, the reason that a impedance of the ultrasound transducer 7 is different in accordance with an applied voltage, or the like, the positive phase ultrasound transmission signal and the negative phase ultrasound transmission signal are shifted in time direction related to each of the transmission timings, which is a criterion for allowing the relationship of the timing between the positive phase and the negative phase in the positive phase/negative phase addition unit 36 to be aligned with each other, in the transmitting/receiving unit 32. Accordingly, an unintentional relative shift occurs in time direction between the positive phase and the negative phase, so that an unintentional shift also occurs in time direction between the positive phase and the negative phase of the ultrasound reception signals due to the ultrasound waves generated by each of the ultrasound transmission signals.

As a result, the fundamental wave component is not canceled out and remains in the synthetic signal. Accordingly, the positive phase/negative phase addition unit 36 according to the first embodiment minimizes, by adding the positive phase ultrasound reception signal that has been subjected to phasing addition to the negative phase ultrasound reception signal that has been subjected to phasing addition by shifting both of the ultrasound reception signals by a predetermined period of time, the intensity of the fundamental wave component, i.e., a residual intensity of the fundamental wave component, in the synthetic signal obtained from the positive phase ultrasound reception signal that has been subjected to phasing addition and the negative phase ultrasound reception signal that has been subjected to phasing addition.

When the positive phase/negative phase addition unit 36 sets the predetermined period of time (for example, at the time of generation of the ultrasound imaging device 3), the positive phase/negative phase addition unit 36 calculates a plurality of synthetic signals by adding each of the positive phase ultrasound echoes received by the transmitting/receiving unit 32 and the negative phase ultrasound echoes received by the transmitting/receiving unit 32 by shifting the ultrasound echoes by different periods of time, sets, to the predetermined period of time, a period of time (hereinafter, also referred to as a “reference shift amount”) that is shifted at the time of calculation of a synthetic signal having the minimum fundamental wave component among the plurality of synthetic signals, and stores the obtained result in the storage unit 39.

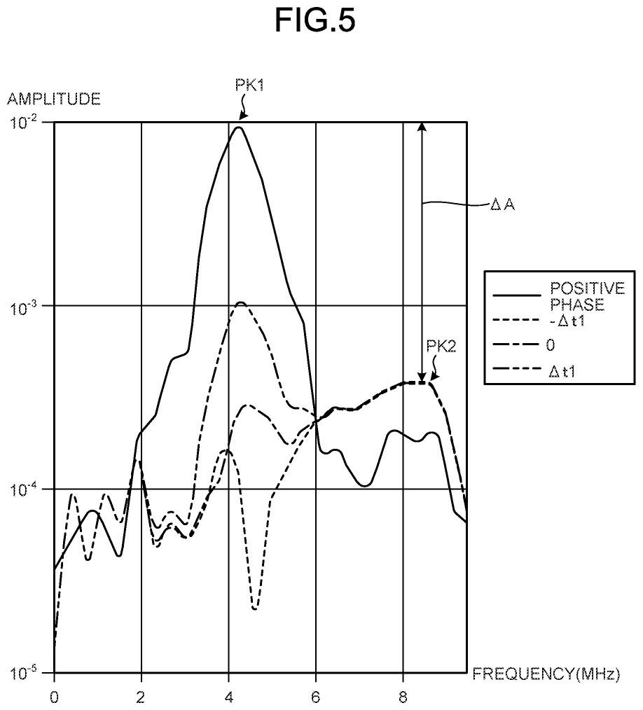

FIG. 5 is a diagram illustrating an addition result that is obtained by changing a shift amount of a period of time between positive phase ultrasound echoes and negative phase ultrasound echoes and that is represented by using a frequency component. The vertical line illustrated in FIG. 5 indicates an amplitude of the ultrasound waves, whereas the horizontal line indicates a frequency (MHz). FIG. 5 illustrates the amplitude of the positive phase and the amplitude of the synthetic signal in the case where the shift amount is defined as -At, 0, and At. A peak PK1 of the fundamental wave component appears in the amplitude of the positive phase indicated by the solid line, whereas a peak PK2 of the harmonic component appears in the amplitude of the addition result (synthetic signal) of the positive phase and the negative phase that are indicated by the lines other than the solid line. In FIG. 5, a difference AA between the peak PK1 of the fundamental wave component and the peak PK2 of the harmonic component is 28 dB. As indicated by the alternate long and short dash line, if addition is performed by without shifting a period of time between the positive phase ultrasound reception signal that has been subjected to phasing addition and the negative phase ultrasound reception signal that has been subjected to phasing addition, it is able to confirm that a fundamental wave component associated with the peak PK1 remains. In contrast, as indicated by the broken line, if a period of time between the positive phase ultrasound reception signal that has been subjected to phasing addition and the negative phase ultrasound reception signal that has been subjected to phasing addition is shifted by an amount -At, the fundamental wave component associated with the peak PK1 is sufficiently decreased with respect to the harmonic component. Furthermore, as indicated by the chain double-dashed line, if a period of time between the positive phase ultrasound reception signal that has been subjected to phasing addition and the negative phase ultrasound reception signal that has been subjected to phasing addition is shifted by an amount At, the residual amount of the fundamental wave component associated with the peak PK1 is further increased as compared to a case in which the time is not shifted. Furthermore, in each of the lines, the intensity of each of the harmonic components associated with the peak PK2 is almost unchanged. In addition, as indicated by the solid line, the amplitude of the harmonic component of the positive phase ultrasound reception signal that has been subjected to phasing addition is not subjected to addition, and thus the size of the amplitude of the harmonic component of the positive phase ultrasound reception signal indicated by the solid line is half the size of the amplitude indicated by the other lines.

Here, if an output of the fundamental wave from the positive phase/negative phase addition unit 36 in the case where the phase between the positive phase ultrasound reception signal subjected to phasing addition and the negative phase ultrasound reception signal subjected to phasing addition is shifted by an amount 60 is denoted by PIf, PIf is able to be represented by a difference between the sine waves as below.

PIf˜sin(ωt+Δθ/2)−sin(ωt-Δθ/2)=−2×cos(ωt)×sin(Δθ) (1)

In contrast, if an output of the harmonic from the positive phase/negative phase addition unit 36 is denoted by PIh, PIh is able to be represented by the sum of the sine waves as below.

PIh˜sin (2 ωt+Δθ/2)+sin (2ωt−Δθ/2)=2×sin (2 ωt)×cos (Δθ) (2)

If Δθ is small enough to be able to approximate zero, a differential result of PIf and PIh are represented below.

dPIf/dΔθ˜dsin(Δθ)/dΔθ≈ (3)

dPIh/dΔθ˜dcos (Δθ)/dΔθ≈0 (4)

Therefore, regarding PIf, a variation amount is in proportion to Δθ when Δθ is in the vicinity of zero, whereas, regarding PIh, a variation amount is approximately zero when Δθ is in the vicinity of zero. As a result, it is possible to control the size (the residual intensity of the fundamental wave component of the synthetic signal added by the positive phase/negative phase addition unit 36) of PIf when Δθ is in the vicinity of zero by using Δθ without influencing the size of PIh (intensity of the harmonic component). Furthermore, by converting a phase difference to a time difference, it is possible to control the residual intensity of the fundamental wave component of the synthetic signal added by the positive phase/negative phase addition unit 36 on the basis of the shift amount of the period of time between the positive phase ultrasound reception signal subjected to phasing addition and the negative phase ultrasound reception signal subjected to phasing addition.

The positive phase/negative phase addition unit 36 sets, to the predetermined period of time, −Δt among the plurality of shift amounts (Δt, 0, and −Δt) illustrated in FIG. 5, and stores the set data in the storage unit 39.

Then, the positive phase/negative phase addition unit 36 reads the predetermined period of time from the storage unit 39 at the time of observation, and adds the positive phase ultrasound reception signal subjected to phasing addition and the negative phase ultrasound reception signal subjected to phasing addition by shifting the ultrasound reception signals by the predetermined period of time.

However, the positive phase/negative phase addition unit 36 may calculate a plurality of synthetic signals by adding, at the time of a start of observation, each of frames, sound rays, each of the positive phase ultrasound reception signal that is subjected to phasing addition and that is input from the phasing addition unit 35 and the negative phase ultrasound reception signal that is subjected to phasing addition and that is input from the phasing addition unit 35 by shifting the ultrasound reception signals by different periods of time, and may set the period of time that is shifted at the time of calculation of a synthetic signal having the minimum fundamental wave component among the plurality of synthetic waves to the predetermined period of time.

Furthermore, the predetermined period of time may sometimes need resolution smaller than a sampling period Ts of the ultrasound reception signal that is an output of the phasing addition unit 35. In this case, as described in Japanese Laid-open patenttent Publication No. 5-15532, when interpolation is performed on a sampling value of the RF data of each of the piezoelectric elements in the time direction by the phasing addition unit 35, it is possible to implement a shift of the predetermined period of time by allowing the predetermined period of time to be included in an interpolation position.

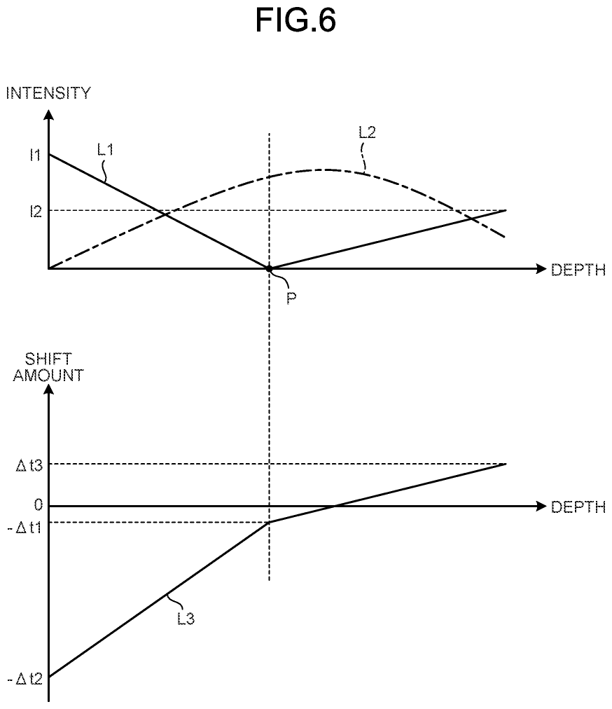

Furthermore, the positive phase/negative phase addition unit 36 may set the predetermined period of time to a different value in accordance with a depth, and may set, at at least one point in a depth direction, the reference shift amount to the predetermined period of time. For example, the positive phase/negative phase addition unit 36 may set, at an observation position that is input by a user by using the input unit 37, the reference shift amount to the predetermined period of time. FIG. 6 is a diagram illustrating a relationship between a depth and an intensity of each of the fundamental wave component and the harmonic component and a relationship between a depth and a shift amount. A line L1 illustrated in FIG. 6 indicates an intensity of the fundamental wave component in the depth direction, a line L2 indicates an estimated intensity of the harmonic component in the depth direction, and a line L3 indicates a shift amount between the positive phase ultrasound reception signal and the negative phase ultrasound reception signal in the depth direction. As illustrated in FIG. 6, the positive phase/negative phase addition unit 36 adds the positive phase ultrasound reception signal and the negative phase ultrasound reception signal by shifting the ultrasound reception signals by the predetermined period of time (reference shift amount=−Δt1), so that it is possible to minimize (substantially zero) the residual intensity of the fundamental wave component at an observation position P.

Furthermore, the positive phase/negative phase addition unit 36 increases a change in the shift amount (the predetermined period of time) between the positive phase ultrasound reception signal and the negative phase ultrasound reception signal up to −Δt2 as the depth approaches a shallower position from the observation position P, and increases the residual intensity of the fundamental wave component up to I1. In other words, the positive phase/negative phase addition unit 36 defines, as the predetermined period of time, the time shifted at the time of calculation of a synthetic wave having a larger fundamental wave component among the plurality of synthetic signals as the depth approaches a shallower position from the observation position P. Similarly, the positive phase/negative phase addition unit 36 increases a change in the shift amount between the positive phase ultrasound reception signal and the negative phase ultrasound reception signal up to Δt3 as the depth approaches a deeper position from the observation position P, and increases the residual intensity of the fundamental wave component up to 12. In other words, the positive phase/negative phase addition unit 36 defines, as the predetermined period of time, the time shifted at the time of calculation of a synthetic wave having a larger fundamental wave component among the plurality of synthetic signals as the depth approaches a deeper position from the observation position P. These processes are performed in order to complement the ultrasound image by increasing the residual intensity of the fundamental wave component because the intensity of the harmonic component is weak in an area closer to the ultrasound transducer 7 and in an area away from the ultrasound transducer 7. In addition, FIG. 6 illustrates an example in which a shift amount of each of the phases is linearly changed; however, the example is not limited to this. For example, the shift amount may be changed along a predetermined curve or may be discretely changed.

Furthermore, in addition to shifting the phases, the positive phase/negative phase addition unit 36 may adjust an intensity of a gain, a filter, or the like in accordance with the depth. The positive phase/negative phase addition unit 36 is implemented by using a CPU, various arithmetic circuits, or the like.

The input unit 37 is implemented by using a user interface, such as a keyboard, a mouse, a touch panel, or trackball, and receives an input of various kinds of information. The input unit 37 receives an input of an observation position performed by the user. The observation position is the most desired position to be observed by the user included in the ultrasound image.

The control unit 38 performs the overall control of the entire of the endoscope system 1. The control unit 38 is implemented by using a CPU, various arithmetic circuits, or the like having an arithmetic function or a control function. The control unit 38 performs the overall control of the ultrasound imaging device 3 by reading information stored in the storage unit 39 from the storage unit 39 and performing various kinds of arithmetic processing related to the actuation method performed by the ultrasound imaging device 3. Furthermore, the control unit 38 is able to be configured by using a CPU or the like that is common to the signal processing unit 33, the image processing unit 34, the phasing addition unit 35, or the positive phase/negative phase addition unit 36.

The storage unit 39 stores therein various programs for operating the endoscope system 1, data including various parameters or the like needed for operating the endoscope system 1, and the like. Furthermore, the storage unit 39 stores therein various programs including an operation program for executing a method of operating the endoscope system 1. The actuation program may be widely distributed by being stored in in a computer-readable storage medium, such as a hard disk, a flash memory, a CD-ROM, a DVD-ROM, a flexible disk. In addition, the various programs described above may be acquired by download via a communication network. The communication network mentioned here is implemented by, for example, an existing public line network, a local area network (LAN), a wide area network (WAN), or the like regardless of a wired or wireless connection.

The storage unit 39 having the configuration described above is implemented by using a read only memory (ROM) in which various programs or the like are installed in advance, a random access memory (RAM) in which arithmetic parameters, data, or the like of each of processes are stored, or the like.

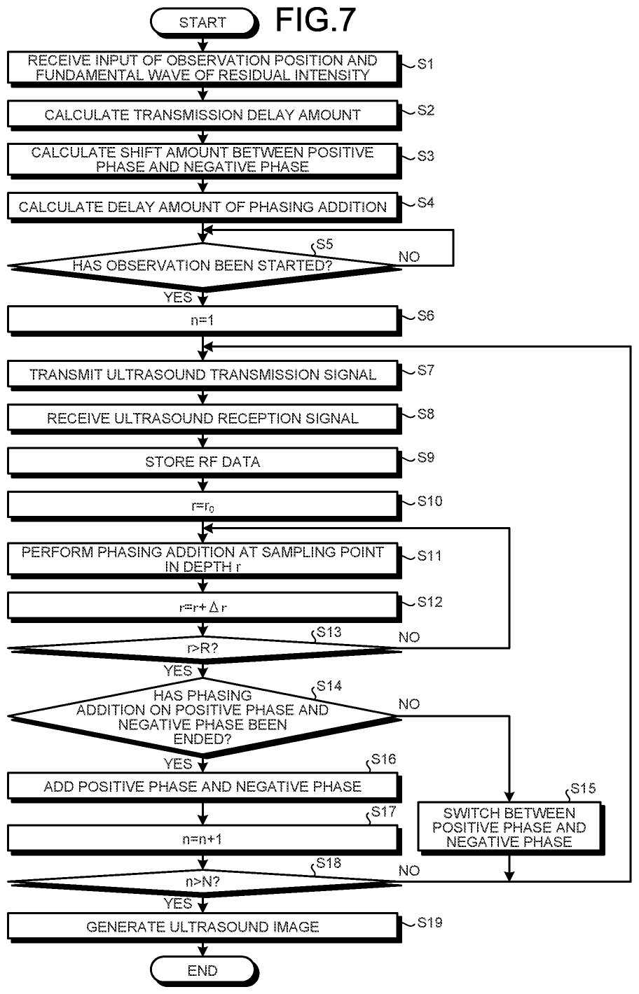

Operation of ultrasound imaging device In the following, an operation of the ultrasound imaging device 3 will be described. FIG. 7 is a flowchart illustrating the overall process performed by the ultrasound signal processing apparatus according to the first embodiment. As illustrated in FIG. 7, first, the input unit 37 included in the ultrasound imaging device 3 receives an input of the observation position P (see FIG. 6) and the residual intensities Il and 12 (see FIG. 6) of the fundamental wave components (Step S1).

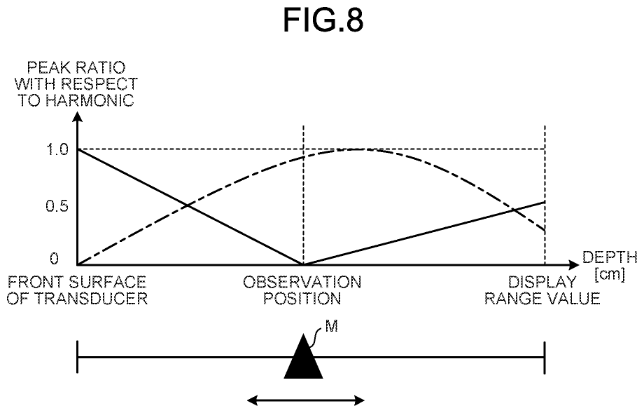

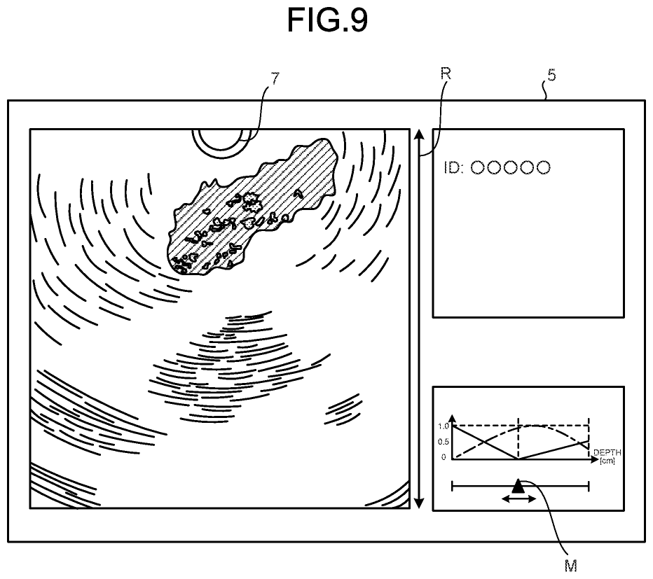

FIG. 8 is a diagram illustrating an example of an input of the observation position. As illustrated in FIG. 8, the user sets the observation position P by operating the input unit 37 and moving the position of a marker M displayed on the display device 5. The vertical axis illustrated in FIG. 8 is able to be defined by, for example, a luminance value of an ultrasound image, and the solid line indicates a standardized value obtained by dividing the luminance value of the fundamental wave component by the luminance value of the harmonic component at the peak. Furthermore, the alternate long and short dash line represents the luminance value of the harmonic component standardized by using the maximum value as 1. Δt the observation position P, the luminance value of the fundamental wave component indicated by the solid line becomes the minimum value (substantially zero). In addition, it is assumed that the residual intensity Il of the fundamental wave component is preset to 1.0, and the residual intensity 12 is preset to 0.5. The observation position P and the residual intensities Il and 12 of the fundamental wave component may be preset and may be stored in the storage unit 39. The horizontal axis illustrated in FIG. 8 represents a depth (cm), which is displayed up to a display range value from a position that is closest to the ultrasound transducer 7.

Subsequently, the control unit 38 calculates an amount of delay with respect to the ultrasound transmission signal that is transmitted to each of the piezoelectric elements included in the ultrasound transducer 7 (Step S2).

Specifically, the control unit 38 calculates an appropriate amount of delay in accordance with the position of each of the piezoelectric elements included in the ultrasound transducer 7.

Furthermore, the control unit 38 calculates, in the positive phase/negative phase addition unit 36, a shift amount of the period of time at the time of addition of the positive phase ultrasound reception signal and the negative phase ultrasound reception signal (Step S3).

Then, the control unit 38 calculates, in the phasing addition performed by the phasing addition unit 35, an amount of delay added to the REF data that is obtained from each of the piezoelectric elements included in the ultrasound transducer 7 in accordance with the distance r from the sampling point SP (Step S4). Here, if resolution that is smaller than the sampling period Ts is needed for the predetermined period of time, the predetermined period of time (the time shift amount between the positive phase and the negative phase) is included in this amount of delay.

After that, the control unit 38 determines whether or not the input unit 37 receives a predetermined operation input associated with a start of observation (Step S5).

If the control unit 38 determines that the input unit 37 does not receive a predetermined operation input associated with a start of observation (No at Step S5), the control unit 38 enters a standby state in which the process at Step S5 is repeated.

In contrast, if the control unit 38 determines that the input unit 37 receives a predetermined operation input associated with a start of observation (Yes at Step S5), the control unit 38 sets a variable n representing a piezoelectric element number such that n=1 (Step S6).

Subsequently, the transmitting/receiving unit 32 transmits, under the control of the control unit 38, the ultrasound transmission signal to each of the piezoelectric elements included in the ultrasound transducer 7 (Step S7).

Furthermore, the transmitting/receiving unit 32 receives the ultrasound reception signal from each of the piezoelectric elements included in the ultrasound transducer 7 (Step S8).

The RF data that is received by the transmitting/receiving unit 32 is stored in the storage unit 39 (Step S9).

Subsequently, the control unit 38 sets the distance r that represents a depth such that r=r₀ (Step S10), where r₀ denotes a radius of a front surface of the ultrasound transducer 7.

Then, the phasing addition unit 35 performs phasing addition of the distance r at the sampling point SP (Step S11).

After that, the control unit 38 sets the distance r such that r=r+Δr (Step S12).

Furthermore, the control unit 38 determines whether or not the distance r is r>R (Step S13). If the control unit 38 determines that the distance r is not r>R (No at Step S13), the process returns to Step S11 and is continued.

Namely, the ultrasound imaging device 3 performs phasing addition on the distances ro to R by performing the processes at Steps S10 to S13 at intervals of Ar. In other words, the ultrasound imaging device 3 is able to generate an ultrasound image in the range of the distances ro to R.

If the control unit 38 determines that the distance r is r>R (Yes at Step S13), the control unit 38 determines whether or not the phasing addition is ended in both of the positive phase and the negative phase (Step S14).

If the control unit 38 determines that the phasing addition is not ended in both of the positive phase and the negative phase (No at Step S14), the control unit 38 switches between the positive phase and the negative phase (Step S15), and returns to Step S7. In other words, the ultrasound imaging device 3 performs the phasing addition by transmitting and receiving the ultrasound waves of the positive phase, and then, performs the phasing addition by transmitting and receiving the ultrasound waves of the negative phase.

If the control unit 38 determines that the phasing addition is ended in each of the positive phase and the negative phase (Yes at Step S14), the positive phase/negative phase addition unit 36 adds the ultrasound reception signal that is in positive phase and that is subjected to phasing addition and the ultrasound reception signal that is in negative phase and that is subjected to phasing addition by shifting the ultrasound reception signals by an amount corresponding to the predetermined period of time (Step S16). However, if a shift of the predetermined period of time has already been performed at Step S4, there is no need to perform the shift process.

After that, the control unit 38 sets the variable n such that n=n+1 (Step S17).

Furthermore, the control unit 38 determines whether or not the variable n is n>N (Step S18). If the control unit 38 determines that the variable n is not n>N (No at Step S18), the process returns to Step S7 and is continued. Here, N is associated with the number of piezoelectric elements included in the ultrasound transducer 7. Accordingly, the ultrasound imaging device 3 adds, by performing the processes at Steps S6 to S18, the positive phase ultrasound reception signals that are subjected to phasing addition and the negative phase ultrasound reception signals that are subjected to phasing addition located at the positions of all of the piezoelectric elements having the assigned piezoelectric element numbers 1 to N by shifting the ultrasound reception signals by the predetermined period of time.

After that, the ultrasound imaging device 3 generates an ultrasound image by using the calculation result obtained from the positive phase/negative phase addition unit 36 and allows the calculation result to be displayed on the display device 5. FIG. 9 is a diagram illustrating one example of the ultrasound image. As illustrated in FIG. 9, the generated ultrasound image is displayed on the display device 5. If the ultrasound transducer 7 is a convex type ultrasound transducer, the ultrasound transducer 7 is located at an upper part of the ultrasound image. A distance R illustrated in FIG. 9 corresponds to a value obtained by adding a radius ro of the ultrasound transducer 7 to a width of the horizontal axis illustrated in FIG. 8 (a distance from a position that is closest to the ultrasound transducer 7 to a position of the display range value). Δt this time, it may be possible to display the observation position P and the values of the residual intensities Il and 12 of the fundamental wave components that are set in FIG. 8 in parallel to the ultrasound image. Furthermore, the observation position P may be configured such that the position of the observation position P is able to be changed by shifting the position of the marker M on the display screen.

As described above, according to the first embodiment, as a result of the positive phase/negative phase addition unit 36 adding the positive phase ultrasound reception signal subjected to phasing addition and the negative phase ultrasound reception signal subjected to phasing addition by shifting the ultrasound reception signals by the predetermined period of time, it is possible to minimize the fundamental wave component at the position that is desired to be observed by the user, so that it is possible to generate an ultrasound image having high resolution due to the harmonic component at the position desired to be observed by the user. Furthermore, in an area closer to the ultrasound transducer 7 and in an area away from the ultrasound transducer 7 with a small intensity of the harmonic component, it is possible to generate an ultrasound image that is easily observed by increasing the residual intensity of the fundamental wave component in accordance with an input or the like performed by the user.

In this way, according to the ultrasound imaging device 3, it is possible to easily adjust the residual intensity of the fundamental wave component.

In addition, the shift amount of the time between the positive phase ultrasound reception signal subjected to phasing addition and the negative phase ultrasound reception signal subjected to phasing addition added in the positive phase/negative phase addition unit 36 may be a value that is preset or may be a fixed value that is in accordance with an input; however, the shift amount of the period of time may be a value calculated from feedback control, prediction control, or control performed by artificial intelligence (AI) in accordance with an instruction of the depth desired to be observed by the user, an observation mode, the characteristic of the received ultrasound reception signal, an analysis of an ultrasound image, or the like.

Furthermore, in the first embodiment described above, a case has been described as an example in which the ultrasound waves are transmitted and received twice, i.e., the ultrasound waves having the positive phase and the ultrasound waves having the negative phase are transmitted and received; however, the number of times the ultrasound waves are transmitted and received is not particularly limited. For example, the ultrasound waves may be transmitted and received four times, i.e., the ultrasound waves having the positive phase are transmitted and received twice and the ultrasound waves having the negative phase are transmitted and receives twice, and then these ultrasound waves may be added.

According to the disclosure, it is possible to implement the ultrasound signal processing apparatus, the method of operating the ultrasound signal processing apparatus, and the program for operating the ultrasound signal processing apparatus capable of appropriately extracting a harmonic component and easily adjusting the residual intensity of the fundamental wave component when the fundamental wave component is appropriately removed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound signal processing apparatus comprising:
a receiver configured to receive a positive phase ultrasound reception signal and a negative phase ultrasound reception signal; and
a processor comprising hardware, the processor being configured to
perform phasing addition on each of the positive phase ultrasound reception signal and the negative phase ultrasound reception signal, and
adding the positive phase ultrasound reception signal subjected to the phasing addition and the negative phase ultrasound reception signal subjected to the phasing addition with shifting of a predetermined period of time.

2. The ultrasound signal processing apparatus according to claim 1, further comprising a storage, wherein
the processor is further configured to
calculate a plurality of synthetic waves by adding the positive phase ultrasound reception signal received by the receiver and the negative phase ultrasound reception signal received by the receiver with shifting of different periods of time,
set, to the predetermined period of time, a first time period that is shifted when calculating a synthetic wave having a minimum fundamental wave component among the plurality of synthetic waves, and
store the first time period in the storage.

3. The ultrasound signal processing apparatus according to claim 2, wherein
the processor is further configured to
set the predetermined period of time to a different period of time in accordance with a depth,
set, to the predetermined period of time, a second time period that is shifted when calculating a synthetic wave having a minimum fundamental wave component among the plurality of synthetic waves at at least one point in a depth direction, and
store the second time period in the storage.

4. The ultrasound signal processing apparatus according to claim 2, further comprising an input circuit configured to input an observation position, wherein
the processor is further configured to
set, to the predetermined period of time, a third time period that is shifted when calculating a synthetic wave having a minimum fundamental wave component among the plurality of synthetic waves at the input observation position, and
store the third time period in the storage.

5. The ultrasound signal processing apparatus according to claim 4, wherein
the processor is further configured to
set, to the predetermined period of time, a fourth time period that is shifted at time of calculation of a synthetic wave having a larger fundamental wave component among the plurality of synthetic waves as a depth approaches a shallower position from the observation position, and store the fourth time period in the storage, and
set, to the predetermined period of time, a fifth time period that is shifted at time of calculation of a synthetic wave having a larger fundamental wave component among the plurality of synthetic waves as a depth approaches a deeper position from the observation position store the fifth time period in the storage.

6. A method of operating an ultrasound signal processing apparatus, the method comprising:
receiving, by a receiver, a positive phase ultrasound reception signal and a negative phase ultrasound reception signal;
performing, by a processor comprising hardware, phasing addition on each of the positive phase ultrasound reception signal and the negative phase ultrasound reception signal; and
adding, by the processor, the positive phase ultrasound reception signal subjected to the phasing addition and the negative phase ultrasound reception signal subjected to the phasing addition with shifting of a predetermined period of time.

7. A non-transitory computer-readable recording medium with an executable program stored thereon, the program causing an ultrasound signal processing apparatus to execute:
receiving, by a receiver, a positive phase ultrasound reception signal and a negative phase ultrasound reception signal;
performing, by a processor comprising hardware, phasing addition on each of the positive phase ultrasound reception signal and the negative phase ultrasound reception signal; and
adding, by the processor, the positive phase ultrasound reception signal subjected to the phasing addition and the negative phase ultrasound reception signal subjected to the phasing addition with shifting of a predetermined period of time.
